(19) United States
(12) Patent Application Publication
(10) Pub. No.: US 2020/0305963 A1
(43) Pub. Date: Oct. 1, 2020

(54) BIPOLAR ELECTROSURGICAL TOOL

(71) Applicant: KARL STORZ SE & Co. KG, Tuttlingen (DE)

(72) Inventors: Sebastian WAGNER, Tuttlingen (DE); Sebastian WENZLER, Tuttlingen (DE); Klaus IRION, Tuttlingen (DE); Axel STICKEL, Tuttlingen (DE); Thomas HINDING, Tuttlingen (DE); Emma HAAF, Tuttlingen (DE)

(21) Appl. No.: 16/832,127

(22) Filed: Mar. 27, 2020

(30) Foreign Application Priority Data
Mar. 28, 2019 (DE) 10 2019 108 140.8

(51) Int. Cl. A61B 18/14

(52) U.S. Cl. CPC A61B 18/1445; A61B 2018/00589

(57) ABSTRACT

A bipolar electrosurgical tool (20) for a bipolar electrosurgical instrument (10) includes a first jaw part (30) with a first gripping face (31), a second jaw part (40) with a second gripping face (41) facing the first gripping face (31), a joint (50) that enables the second jaw part (40) to move pivotally in relation to the first jaw part (30), a first electrode (63) on the first jaw part (30) and a second electrode (64) on the first jaw part (30). The second electrode (64) is electrically insulated from the first electrode (63). There is no electrode arranged on the second jaw part (40).

### CROSS REFERENCE TO RELATED APPLICATIONS

This application claims the benefit of priority under 35 U.S.C. § 119 of German Application 10 2019 108 140.8, filed Mar. 28, 2019.

### TECHNICAL FIELD

The present invention relates to a bipolar electrosurgical tool and a bipolar electrosurgical instrument.

### TECHNICAL BACKGROUND

In all surgical procedures, it is important to avoid bleeding. In the case of microinvasive operations, bleeding entails particular risks, since it can impair the view of the operating staff, and haemostasis can be more difficult than in open surgery. Safely sealing blood vessels—for example before they are transected—is thus an ongoing subject for research and development in medical technology. The same applies to the safe sealing of other hollow organs.

In electrosurgery, electrical currents heat tissue in order to coagulate it. This is frequently also called cauterising or fusing. In this case, in particular colloidal substances transition from a solution state to a gel state. In the event of heating to too high a temperature, however, the affected tissue may be damaged to such an extent that safe, durable sealing and rapid healing are no longer ensured.

The success of an electrosurgical measure depends on the type and condition of the tissue, the current density and its dependence on location, the duration of current flow, the temperature reached and its dependence on location, the mechanical pressure and other parameters.

The objectives, when developing electrosurgical tools and instruments, are to arrive at a process that can be regulated, by open or closed-loop control, as precisely as possible, with defined regions heated to temperatures that are as defined as possible, and hence to achieve as predictable a quality of the vessel sealing as possible, or in more general terms the change in tissue, and to prevent tissue from adhering to the tool or instrument. Further, electrosurgical tools should be as small as possible but also mechanically robust, and also shaped in a manner making them suitable for dissection.

EP 1 632 192 A1 discloses an instrument for the sealing of vessels that has two gripping jaws that are movable relative to one another (paragraphs [0011], [0018]). Each gripping jaw includes a pair of electrically conductive, spaced-apart vessel sealing surfaces extending along the gripping jaw (op. cit.). Each pair of vessel sealing surfaces is connected to a source of electrosurgical energy (op. cit.). The gripping jaws may take a form such that they are configured as mirror images of one another, or are not configured as mirror images of one another (paragraphs [0059], [0060], [0066], [0067]).

### SUMMARY

It is an object of the present invention to provide an improved bipolar electrosurgical tool and an improved bipolar electrosurgical instrument.

A bipolar electrosurgical tool for a bipolar electrosurgical instrument includes a first jaw part with a first gripping face, a second jaw part with a second gripping face facing the first gripping face, a joint that enables the second jaw part to move pivotally in relation to the first jaw part, a first electrode on the first jaw part, and a second electrode on the first jaw part, wherein the second electrode is electrically insulated from the first electrode.

A bipolar electrosurgical tool for a bipolar electrosurgical instrument includes a first jaw part with a first gripping face, a second jaw part with a second gripping face facing the first gripping face, a joint that enables the second jaw part to move pivotally in relation to the first jaw part, a first electrode on the first jaw part, and a second electrode on the first jaw part or on the second jaw part, wherein the second electrode is electrically insulated from the first electrode, and wherein the second gripping face is electrically insulating where it is opposite the first electrode.

In other words, there is no conductive region or sub-region of the second gripping face—not even partly or overlapping—opposite the first electrode. The term “opposite” relates in particular to a closed position of the bipolar electrosurgical tool in which the gripping faces on the jaw parts are at the minimum possible spacing and are opposite one another at a small spacing or are partly or entirely in contact. A first point on a first surface is opposite a second point on a second surface if it lies on a surface normal to the second surface at the second point. In the event of there being one or two curved gripping faces, there is taken as the reference surface to which the surface normal relates in particular a planar surface that is parallel to the pivot axis defined by the joint and parallel to the principal directions of extent of the jaw parts.

In a bipolar electrosurgical tool as described here, either the second electrode is arranged on the first jaw part and the second gripping face is electrically insulating where it is opposite the second electrode; or the second electrode is arranged on the second jaw part and the first gripping face is electrically insulating where it is opposite the second electrode.

Either the first electrode is arranged on the first jaw part and there is no conductive region or sub-region of the second gripping face—not even partly or overlapping—opposite the first electrode. Or the first electrode is arranged on the second jaw part and there is no conductive region or sub-region of the first gripping face—not even partly or overlapping—opposite the first electrode.

A bipolar electrosurgical tool for a bipolar electrosurgical instrument includes a first jaw part with a first gripping face, a second jaw part with a second gripping face facing the first gripping face, a joint that enables the second jaw part to move pivotally in relation to the first jaw part, a first electrode on the first jaw part, and a second electrode on the first jaw part, wherein the second electrode is electrically insulated from the first electrode, and wherein there is no electrode arranged on the second jaw part.

The bipolar electrosurgical instrument is in particular part of a microinvasive instrument (for example for laparoscopy), or is provided and configured to form a microinvasive or other electrosurgical instrument together with one or more other components. The bipolar electrosurgical tool is permanently and mechanically connected for example to a distal end of a shaft of an instrument—that is to say that, without a tool, or using the devices available to medical staff, is it is not reversibly and non-destructively separable from the shaft. As an alternative, the bipolar electrosurgical instrument may be connectable to a distal end of a shaft of an instrument detachably—that is to say that it can be detached and connected again non-destructively, using the devices available to medical staff. In both cases, in particular part of the bipolar electrosurgical tool is mechanically rigidly connected or connectable to the distal end of the shaft.

The first gripping face on the first jaw part is formed in particular by the entire surface region of the first jaw part facing the second jaw part. The second gripping face on the second jaw part is formed in particular by the entire surface region of the second jaw part facing the first jaw part. Both gripping faces may be smooth or substantially smooth, planar or curved, and may have a profile and/or grooves, webs or concave or convex regions facilitating reliable gripping and holding. The bipolar electrosurgical tool is in particular configured such that both gripping faces can be in point, linear or surface contact with one another when no tissue is located between the gripping faces.

The joint is arranged in particular between the proximal end of the second jaw part and the proximal end of the first jaw part. The joint defines a pivot axis that is in particular at a right angle or substantially at a right angle to the longitudinal axis of a shaft connected in the manner provided to the bipolar electrosurgical tool and/or at a right angle to the directions of maximum extent of the jaw parts. When used as intended, the pivot axis defined by the joint is in particular parallel or substantially parallel to a longitudinal direction of a hollow organ to be sealed using the bipolar electrosurgical tool, and hence for example to the direction of blood flow in a blood vessel to be sealed.

The first electrode and the second electrode on the first jaw part are in particular each in the form of a strip and extend next to one another, parallel or substantially parallel to the longitudinal direction of the first jaw part. For this reason, current flowing between the first electrode and the second electrode through tissue held between the jaw parts flows substantially parallel to the gripping faces, at a right angle to the longitudinal directions of the jaw parts and the electrodes, and hence substantially parallel to a longitudinal direction of a hollow organ held by the bipolar electrosurgical tool and for example to the direction of blood flow of a blood vessel held by the bipolar electrosurgical tool. The resulting distribution of current density in the tissue held by the bipolar electrosurgical tool may simplify or enable concentration of the heating of tissue on the desired region and an improvement in the achievable or achieved quality of sealing of a hollow organ.

Arranging electrodes exclusively on the first jaw part can markedly simplify the layout and manufacture of the second jaw part. It is also possible to simplify the layout and manufacture of the joint, since there is no need for an electrically conductive supply line, which is nonetheless electrically insulated from other components, to an electrode on the second jaw part. Further, the risk of tissue adhering to the second jaw part is markedly reduced, as a result of which the bipolar electrosurgical tool can be used more simply and more safely. Further, the fact that the second jaw part is constructed more simply can facilitate a more rigid mechanical layout and hence the transmission of larger forces to tissue that is held by the bipolar electrosurgical tool.

In a bipolar electrosurgical tool as described here, in particular the first jaw part is mechanically rigidly connected or rigidly connectable to a distal end of a shaft of an electrosurgical instrument.

The first jaw part is in particular configured as an inherently rigid unit with a proximal end of the bipolar electrosurgical tool, wherein the proximal end of the bipolar electrosurgical tool is mechanically rigidly connected or rigidly connectable to a distal end of a shaft. Thus, during use of the bipolar electrosurgical tool as intended, only the second jaw part is pivoted in relation to the shaft of the instrument. The first jaw part remains unmoved in relation to the shaft of the instrument, in a position that extends the length of the shaft, in particular in a straight line.

The fact that the first jaw part is mechanically rigidly connected to the shaft prevents electrical power from being supplied through a joint. Thus, it is possible to avoid resilient electrical lines or sliding contacts and the risks and disadvantages associated with these. The risks and disadvantages of supplying electrical power via a resilient electrical line or a sliding contact that this avoids include material fatigue, abrasion, corrosion and contact resistance.

Further, mechanically rigidly connecting the first jaw part to the distal end of a shaft can enable the layout to be mechanically more robust and simpler. The fact that a joint between the first jaw part and the shaft is dispensed with can, as a result of a simplified layout, also make simpler and more thorough cleaning possible.

In a bipolar electrosurgical tool as described here, in particular the entire second gripping face is configured to be electrically insulating.

In a bipolar electrosurgical tool as described here, in particular the entire surface of the second jaw part is configured to be electrically insulating.

The fact that the entire second gripping face on the second jaw part or the entire outer surface of the second jaw part is configured to be electrically insulating can reduce the risk that undesired current paths are formed through the second jaw part, which may jeopardise the success of an electrosurgical measure.

In a bipolar electrosurgical tool as described here, in particular one or more electrically insulating regions or portions of the first jaw part and/or the second jaw part may be made in particular partly or entirely from ceramic, glass, synthetic material, an elastomer or metal that is coated, in particular with an electrically insulating coating.

A bipolar electrosurgical tool as described here further includes in particular a groove in the first gripping face.

A bipolar electrosurgical tool as described here further includes in particular a groove in the second gripping face.

A bipolar electrosurgical tool as described here further includes in particular a cutting device that can be moved between the first jaw part and the second jaw part, for the purpose of mechanically transecting tissue that is held between the first jaw part and the second jaw part.

The cutting device can be moved in particular in the longitudinal direction of the jaw parts. The edges of the cutting device extending parallel to the intended direction of movement can engage in a groove in the first gripping face and/or a groove in the second gripping face, and thus be guided mechanically. A blade at the distal end of the cutting device may be arranged in a straight line and at the same time at a right angle to the intended direction of movement, or may be arranged obliquely thereto, in order to facilitate the transection of tissue. As an alternative, the blade may be curved or arched, in which case it runs in particular at least partly obliquely—that is to say not at a right angle to the intended direction of movement. The blade of the cutting device extends in particular substantially from the first jaw part to the second jaw part.

A bipolar electrosurgical tool as described here includes in particular a cutting wire or a cutting wire loop as a monopolar or bipolar electrosurgical cutting device that is arranged between the first jaw part and the second jaw part, for the purpose of transecting by electrosurgery tissue held between the first jaw part and the second jaw part.

The cutting wire or cutting wire loop can be moved in particular in relation to both jaw parts. The cutting wire or cutting wire loop can be moved in particular in a direction parallel to the longitudinal direction of the jaw parts. As an alternative or in addition, the cutting wire or cutting wire loop may be movable in a direction at a right angle to the longitudinal direction of the closed jaw parts.

For example, the cutting wire is rigid at a distal end of a jaw part—that is to say it is secured such that it is not movable in its longitudinal direction—and in a rest position it adopts a form that is V or U-shaped in a bow or a circle or with a sharp angle. In this arrangement, the cutting wire can abut against a concave surface region of the jaw part or be partly or entirely concealed within a groove in a concave surface region of the jaw part. Mechanically pulling on a proximal region of the cutting wire can tension the cutting wire, which can thus be changed from its V or U-shaped form into a straight form until it abuts or almost abuts against the opposing jaw part. In this arrangement, it can transect tissue between the jaw parts by electrosurgery.

The electrosurgical cutting device is in particular configured as a monopolar electrosurgical cutting device, with a large-surface neutral electrode on the outside of the patient's body closing the circuit. As an alternative or in addition, the first electrode and/or the second electrode can be used as the counter-electrode.

Once a blood vessel or another hollow organ has been sealed by electrosurgery, a cutting device between the jaw parts can enable it to be transected. The sealing and transection can thus be performed particularly reliably and in particularly rapid succession.

In a bipolar electrosurgical tool as described here, in particular at least either the first electrode or the second electrode or an electrically insulating surface region of the first jaw part or the second gripping face of the second jaw part is partly or completely provided with a coating that reduces the mechanical adhesion of tissue.

A coating that prevents or reduces the adhesion of tissue can make handling and use of the bipolar electrosurgical tool safer and more reliable.

The first electrode and the second electrode are in particular surface regions on electrode components made from a material of which the thermal conductivity is as high as possible, in particular greater than 10 W/(m·K) (for example steel), or greater than 100 W/(m·K) (for example silver, gold, or electrically conductively doped or coated diamond).

Good dissipation of heat from the electrode, as a result of an electrode component with high thermal conductivity, can counter the heating of tissue abutting against the electrode and, resulting from this, adhesion of the tissue to the electrode.

In a bipolar electrosurgical tool as described here, the first electrode and the second electrode are arranged in particular next to one another and parallel to one another on the first gripping face.

The first electrode and the second electrode are each in particular substantially in the form of a strip or a narrow rectangle, and extend parallel to the principal direction of extent of the first jaw part. The fact of both electrodes being arranged next to one another and parallel to one another can make it possible for the direction of current flow in the tissue of a hollow organ that is to be sealed by electrosurgery to be parallel to the first gripping face and at a right angle to the cross section of the hollow organ. The distributions of current density that can be achieved in this way can enable the hollow organ to be sealed particularly rapidly and particularly safely, in particular without tissue adhering to the electrodes.

In the case of a bipolar electrosurgical tool as described here, the first electrode and the second electrode are arranged in particular on or close to longitudinal edges of the first gripping face that face away from one another, wherein the first gripping face between the first electrode and the second electrode is configured to be completely electrically insulating. The longitudinal edges are those edge portions of the gripping face that extend parallel to one another and to the principal direction of extent of the first jaw part and thus at a right angle to the pivot axis.

The arrangement of the electrodes on or close to the longitudinal edges of the first gripping face and the completely electrically insulating configuration of the gripping face between the electrodes enables a large spacing between the electrodes, with the result that the current flowing through and heating the tissue can flow over a comparatively large distance in the tissue. A hollow organ can thus be closed in a comparatively wide strip or other tissue can be sealed in a comparatively wide strip.

In a bipolar electrosurgical tool as described here, when used as intended the surface area of the first electrode and the surface area of the second electrode are each in particular greater than the cross section of tissue that is gripped or squeezed between the jaw parts in the manner intended during the electrosurgical treatment intended.

In a bipolar electrosurgical tool as described here, when used as intended the width of the first electrode in strip form and the width of the second electrode in strip form are in particular each greater than the thickness of tissue that is gripped or squeezed between the jaw parts in the manner intended during the electrosurgical treatment intended.

Use of the bipolar electrosurgical tool as intended, and the maximum cross sectional surface area and thickness of tissue that is gripped and squeezed between the jaw parts in the manner provided, are in particular unambiguously defined by the registration procedure and the specification of the bipolar electrosurgical tool.

If the surface areas or widths of both electrodes are greater than or markedly (in particular by at least 20% or 50% or by a factor of 2, 3, 5, 10 or 20) greater than the cross sectional surface area or thickness of the gripped and/or squeezed tissue, the current density in the gripped and squeezed tissue is greater by a corresponding factor than in the tissue abutting against the electrodes. Thus, heating of and change in the tissue can be largely restricted to the gripped and squeezed region. Since tissue abutting against the electrodes is not heated or changed, or is at most slightly heated and changed, it is also possible for adhesion of tissue to the electrodes to be prevented or markedly reduced.

In a bipolar electrosurgical tool as described here, the width of the first electrode in strip form and the width of the second electrode in strip form is in each case in particular in the range from 0.3 mm to 5 mm.

When a bipolar electrosurgical tool as described here is used as intended, the thickness of tissue that is gripped or squeezed between the jaw parts in the manner provided is in particular in the range from 0.05 mm to 0.4 mm during the electrosurgical treatment provided.

A bipolar electrosurgical tool as described here further includes in particular a convex surface region between the first electrode and the second electrode, which projects in the direction of the second jaw part.

The convex surface region is for example in the shape of a web or bead having a substantially rectangular, trapezoidal or rounded cross section. The convex surface region is in particular configured to be electrically insulating.

The convex surface region can make it possible to compress or squeeze tissue in a region between the electrodes. As a result, the current density in the compressed or squeezed region may be substantially higher than at the electrodes. As a result, the electrosurgical action on the tissue region that is compressed or squeezed by the convex surface region can be limited, and the adhesion of tissue to the electrodes can be prevented or reduced.

The geometry of the convex surface region substantially defines a sealing region or sealing strip in which, as a result of compression of the tissue, the current density is so high and consequently the tissue is heated to such an extent that it fuses or is cauterised or sealed. The width of the convex surface region or a flat area of the convex surface region substantially defines the width of this sealing region. In the frequent event that the tissue is transected, mechanically or by electrosurgery, after fusion or cauterisation or sealing, a width of the sealing region on each side of the subsequent cut in the order of magnitude of 1 mm (in particular between 0.8 mm and 1.2 mm) has proved useful, in particular if the tissue is a blood vessel or another hollow organ that is to be sealed reliably. The total width of the convex surface region, or a flat area of the convex surface region, is thus in particular between 1.6 mm and 2.4 mm.

A bipolar electrosurgical tool as described here further includes in particular a resilient region on the first gripping face between the first electrode and the second electrode.

In a bipolar electrosurgical tool as described here, the convex surface region is formed in particular by a resilient component.

A resilient region on the first gripping face, and in particular a resilient convex surface region between the electrodes, can enable a resilient adaptation to tissue that is gripped or squeezed by the tool. This adaptation can promote a uniform distribution of pressure in the tissue and prevent a local overloading of the tissue.

In a bipolar electrosurgical tool as described here, the convex surface region is formed in particular by a component comprising silicone, an elastomer or another synthetic material, ceramic, glass or metal that is coated, in particular with an electrically insulating coating.

The convex surface region is formed in particular by a component made from silicone (silicone rubber, silicone elastomer or silicone resin) having a Shore A hardness in the range from 60 to 80. The resilient properties of silicone are readily reproducibly adjustable within a broad range. Moreover, silicone can have sufficient dielectric strength, in the order of magnitude of 20 kV/mm

In a bipolar electrosurgical tool as described here, the convex surface region is formed in particular by a component comprising a material with relatively low thermal conductivity.

The thermal conductivity of the component forming the convex surface region is markedly less than 1 W/(m·K), in particular less than 0.25 W/(m·K), and preferably at most 0.2 W/(m·K) or at most 0.15 W/(m·K) or at most 0.1 W/(m·K).

Low thermal conductivity reduces the dissipation of heat from the tissue region that is compressed by the convex surface region and treated by electrosurgery, and can thus contribute to concentrating the electrosurgical effect on the compressed region.

In a bipolar electrosurgical tool as described here, at least either the first electrode or the second electrode is spaced apart from the convex surface region.

Spacing the electrodes apart from the convex surface region can have the effect that the tissue has a markedly larger cross section in the region of the electrodes than in the area compressed by the convex surface region. The current density and the electrosurgical effect can thus be concentrated on the region compressed by the convex surface region and be markedly less at the electrodes. This can markedly improve the reliability and quality of the electrosurgical measure and reduce the risk of tissue adhering to the electrodes.

In a bipolar electrosurgical tool as described here, the convex surface region includes in particular a blade for mechanically transecting tissue.

The convex surface region and the blade are in particular configured such that, when a first, relatively low predetermined retaining force is exerted, it is possible to seal a hollow organ by electrosurgery, and under a second, relatively high predetermined retaining force it is possible to mechanically transect the previously sealed hollow organ.

In a bipolar electrosurgical tool as described here, the convex surface region is in particular configured in the shape of a roof

A roof-shaped configuration with an edge that projects furthest towards the second jaw part and two surfaces that fall away on either side of the edge can enable tissue to be squeezed such that its cross section or thickness on either side is continuously reduced as far as the edge and is at a minimum at the edge. The result is a current density that increases towards the edge, on either side of the edge. This current density can bring about a temperature distribution that, on either side of the edge, at spacings from the edge that are predetermined or depend on the respective conditions of an electrosurgical measure, can bring about coagulation and sealing of a hollow organ and, in the region of the edge, can bring about heating that is strong enough for the tissue to be transected in the region of the edge.

A bipolar electrosurgical tool as described here further includes in particular a convex surface region on the second gripping face of the second jaw part, wherein the convex surface region projects towards the first gripping face of the first jaw part.

The convex surface region on the second gripping face of the second jaw part can have similar or the same properties, features and functions as the convex surface region on the first gripping face of the first jaw part that has been described. The bipolar electrosurgical tool can either have a convex surface region only on the first gripping face on the first jaw part or only on the second gripping face on the second jaw part, or it can have a respective convex surface region on both gripping faces on both jaw parts. If both gripping faces on both jaw parts each have a convex surface region, then the cross sections thereof may be configured to be mirror symmetrical or different. Further, the convex surface regions can be made from components of the same or different materials.

In a bipolar electrosurgical tool as described here, the convex surface region on the second gripping face of the second jaw part is in particular arranged and configured such that it cannot come into contact with either the first electrode or the second electrode, even in the closed condition of the tool.

Thus, the tissue region that is compressed or squeezed by the convex surface region on the second gripping face of the second jaw part does not reach as far as the electrodes. Tissue that is gripped and squeezed by the tool can thus be less compressed in the vicinity of the electrodes than in the region of the convex surface region. For this reason, the cross sections of the tissue are markedly larger in the region of the electrodes than in the convex surface region. The result is a smaller current density at the electrodes and a higher current density in the tissue region compressed by the convex surface region.

In a bipolar electrosurgical tool as described here, the convex surface region on the second gripping face of the second jaw part is formed in particular by a component made of a resilient material.

A bipolar electrosurgical tool as described here further includes in particular a sensor, for detecting a temperature or a mechanical pressure or a colour or a light intensity of reflected or transmitted light or another physical variable, in order to control or monitor or check an electrosurgical measure or the operational result thereof.

The sensor can be provided and configured for the purpose of detecting a light spectrum, for example a spectrum of reflected or transmitted light, and/or for the purpose of detecting light in one or more predetermined wavelengths or in one or more narrow wavelength ranges. Open or closed-loop control or monitoring of an electrosurgical measure by a sensor can markedly improve the reliability of the method and the reproducibility of the operational result.

A bipolar electrosurgical instrument includes a shaft and a bipolar electrosurgical tool as described here, wherein the bipolar electrosurgical tool is connected or connectable to a distal end of the shaft.

The bipolar electrosurgical tool is in particular mechanically rigidly connected or connectable to the distal end of the shaft. The mechanical connection between the bipolar electrosurgical tool and the distal end of the shaft can be permanent—that is to say that it is not non-destructively detachable using the devices available to medical staff—or it may be detachable reversibly and non-destructively, using the devices available to medical staff.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side schematic view of a distal end of a bipolar electrosurgical instrument;

FIG. 2 is a side schematic view of the distal end of the bipolar electrosurgical instrument from FIG. 1;

FIG. 3 is a schematic cross sectional view of the bipolar electrosurgical tool of the instrument from FIGS. 1 and 2;

FIG. 4 is a schematic side view of a distal end of a further bipolar electrosurgical instrument;

FIG. 5 is a schematic cross sectional view of a further bipolar electrosurgical tool;

FIG. 6 is a schematic cross sectional view of a further bipolar electrosurgical tool;

FIG. 7 is a schematic side view of a tool of a further bipolar electrosurgical instrument;

FIG. 8 is a schematic side view of a tool of a further bipolar electrosurgical instrument;

FIG. 9 is a schematic side view of the tool from FIGS. 8; and

FIG. 10 is a schematic cross sectional view of the tool from FIGS. 8 and 9.

### DESCRIPTION OF PREFERRED EMBODIMENTS

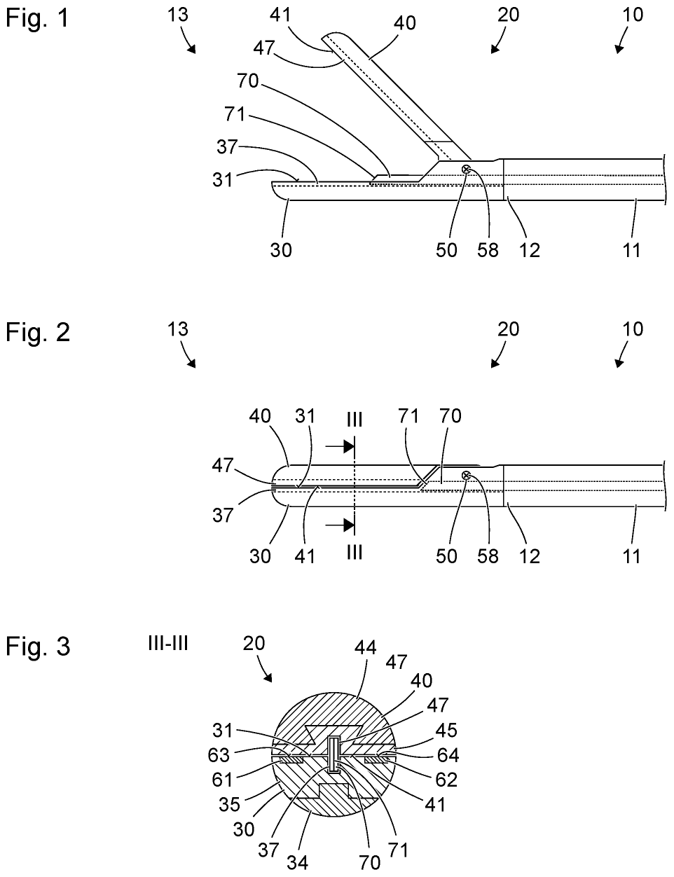

Referring to the drawings, FIG. 1 shows a schematic illustration of a distal end 13 of a bipolar electrosurgical instrument 10. The bipolar electrosurgical instrument 10 has a shaft 11, which may be configured to be straight or curved, rigid or flexible. A proximal end of the shaft 11 is connected or connectable to a handling device, which is not illustrated in FIG. 1. A distal end 12 of the shaft 11 is permanently connected, or may be non-destructively detachably connected, to a bipolar electrosurgical tool 20. The bipolar electrosurgical tool 20 forms the distal end 13 of the bipolar electrosurgical instrument 10.

The bipolar electrosurgical tool 20 includes a first limb or first jaw part 30 with a first gripping face 31, and a second limb or second jaw part 40 with a second gripping face 41. The first gripping face 31 on the first jaw part 30 faces the second jaw part 40. The second gripping face 41 on the second jaw part 40 faces the first jaw part 30. The proximal end of the second jaw part 40 is pivotally connected to the rest of the bipolar electrosurgical tool 20 by way of a joint 50. The joint 50 defines a pivot axis 58 at a right angle to the longitudinal axis of the shaft 11, at a right angle to the plane of the drawing in FIG. 1, and at a right angle to the principal directions of extent of the jaw parts 30, 40.

Arranged in the shaft 11 is a force transmission device (not illustrated in FIG. 1) that extends from the proximal end of the shaft 11 to the bipolar electrosurgical tool 20. The distal end of the force transmission device is coupled to the second jaw part 40 such that a movement of the force transmission device exerted on the handling device (which is not illustrated in FIG. 1) brings about a pivotal movement of the second jaw part 40, about the pivot axis 58 defined by the joint 50, in relation to the distal end 12 of the shaft 11 and the first jaw part 30.

FIG. 1 shows the bipolar electrosurgical tool 20 in an open position. Starting from the open position shown in FIG. 1, the second jaw part 40 can be moved towards the first jaw part 30. This enables tissue to be gripped, held or squeezed between the jaw parts 30, 40.

The bipolar electrosurgical tool 20 includes a groove 37 in the first gripping face 31 on the first jaw part 30, and a groove 47 in the second gripping face 41 on the second jaw part 40. Both grooves 37, 47 are not themselves visible in the illustration in FIG. 1. For this reason, only the contours of the grooves are indicated in FIG. 1, by broken lines.

The bipolar electrosurgical instrument 10 includes a scalpel 70, which can be moved in the grooves 37, 47 even when the jaw parts 30, 40 abut against one another. Here, the scalpel 70 is guided by the grooves 37, 47. A part of the scalpel 70 that projects out of the groove 37 in the first gripping face 31 on the first jaw part 30 is visible in FIG. 1, illustrated by a solid line. A part of the scalpel 70 that is concealed in the groove 37 in the first jaw part 30 is illustrated in a dotted line in FIG. 1.

In FIG. 1, the scalpel 70 is illustrated in dotted lines, extended in the proximal direction to indicate that the scalpel 70 can be moved in a manner controlled from the proximal end of the instrument 10. However, the mechanical coupling between a handling device of the instrument 10 and the scalpel 70 may be made not only by directly extending the component forming the scalpel 70 in the proximal direction but also in another manner.

The scalpel 70 has a blade 71 that, in the illustrated example, is inclined in relation to the direction of movement intended for the scalpel 70. In FIG. 1, the scalpel 70 and the blade 71 are in a position in which, were the jaw parts 30, 40 closed, the scalpel 70 would already have partly transected tissue grasped between them. The scalpel 70 is more clearly visible in FIG. 1 in this position. When the bipolar electrosurgical tool 20 is used as intended, however, the scalpel 70 remains in a predetermined proximal position in which the blade 71 is not exposed and cannot come into contact with any tissue until the jaw parts 30, 40 are closed.

FIG. 2 shows a further schematic illustration of the distal end 13 of the bipolar electrosurgical instrument 10 illustrated in FIG. 1. The type of illustration in FIG. 2 is the same as that in FIG. 1. In FIG. 2, however, the bipolar electrosurgical tool 20 is illustrated in a position different from the position illustrated in FIG. 1.

In the position of the bipolar electrosurgical tool 2 illustrated in FIG. 2, the jaw parts 30, 40 are closed, and the gripping faces 31, 41 of the jaw parts 30, 40 are in contact with one another or are opposite each other at a small spacing. Further, in the position or situation shown in FIG. 2, the scalpel 70 is in the described predetermined proximal position in which the blade 71 is not exposed.

In the position or situation illustrated in FIG. 2, tissue—such as a blood vessel or another hollow organ—can be grasped between the jaw parts 30, 40 and compressed or squeezed. Using the bipolar electrosurgical tool 20 as described below with reference to FIG. 3, this tissue can be changed by electrosurgery, for example to seal the hollow organ. Thereafter, the scalpel 70 can be moved from the position shown in FIG. 2 in the distal direction, in order to transect the tissue.

FIG. 3 shows a schematic illustration on a larger scale of a section along the plane of section III-III indicated in FIG. 2, through the bipolar electrosurgical tool 20 shown in FIG. 2. The plane of section III-III in FIG. 3 is at a right angle to the planes of the drawing of FIGS. 1 and 2 and at a right angle to a principal direction of extent of the jaw parts 30, 40.

In FIG. 3, the cross sections of the jaw parts 30, 40, each substantially semi-circular, are visible. The first jaw part 30 is formed from two components 34, 35, which are mechanically connected permanently and rigidly, in particular by welding or gluing or in another substance-to-substance bond. In the example illustrated, the first component 34, which is spaced apart from the first gripping face 31, is made from metal, and the second component 35, which forms the gripping face 31, is made from an electrically insulating material.

In the second component 35 of the first jaw part 30, the groove 37 that has already been described with reference to FIGS. 1 and 2 is visible in cross section.

Further, adjoining the first gripping face 31, two electrode components 61, 62 are embedded in the electrically insulating second component 35. Exposed surface regions of the electrode components 61, 62 form electrode faces 63, 64. The electrode components 61, 62 and the electrode faces 63, 64 they form are each configured substantially in the form of a strip and are arranged parallel to one another and to the principal direction of extent of the first jaw part 30.

When the electrode faces 63, 64 are in direct contact with tissue, they enable electrical current to move across from the first electrode component 61 into the tissue and from the tissue into the second electrode component 62, or vice versa. Electrical current flowing between the electrode components 61, 62 through tissue between the gripping faces 31, 41 flows substantially parallel to the gripping faces 31, 41 of the jaw parts 30, 40 and at a right angle to the principal directions of extent of the jaw parts 30, 40.

The electrode components 61, 62 are made from a material with as great a thermal conductivity as possible, in particular greater than 10 W/(m·K) (for example steel), or greater than 100 W/(m·K) (for example silver, gold, or electrically conductively doped or coated diamond). Good thermal conductivity of the electrode components 61, 62 enables good heat dissipation away from the electrode faces 63, 64. For this reason, the temperatures of the electrode faces 63, 64 and tissue abutting against the electrode faces 63, 64 remain low. This reduces the likelihood of tissue adhering to the electrode faces 63, 64.

In the illustrated example, the second jaw part 40 is likewise formed from two components 44, 45. In the illustrated example, the components 44, 45 of the second jaw part 40 are mechanically connected to one another form-fittingly, in particular by dovetail-shaped cross sections. As an alternative or in addition, the components 44, 45 of the second jaw part 40 may be connected to one another by a substance-to-substance bond, for example by welding or gluing.

The above-mentioned groove 47 in the second gripping face 41 on the second jaw part 40 is formed exclusively in the second component 45 of the second jaw part 40. It can be seen in FIG. 3 that the scalpel 70 with the blade 71 is guided in the grooves 37, 47 in the jaw parts 30, 40 with low play and low friction.

In the illustrated example, the first component 44 of the second jaw part 40 is made from metal, and the second component 45 of the second jaw part 40 is made from an electrically insulating material. The second jaw part 40 has no electrodes.

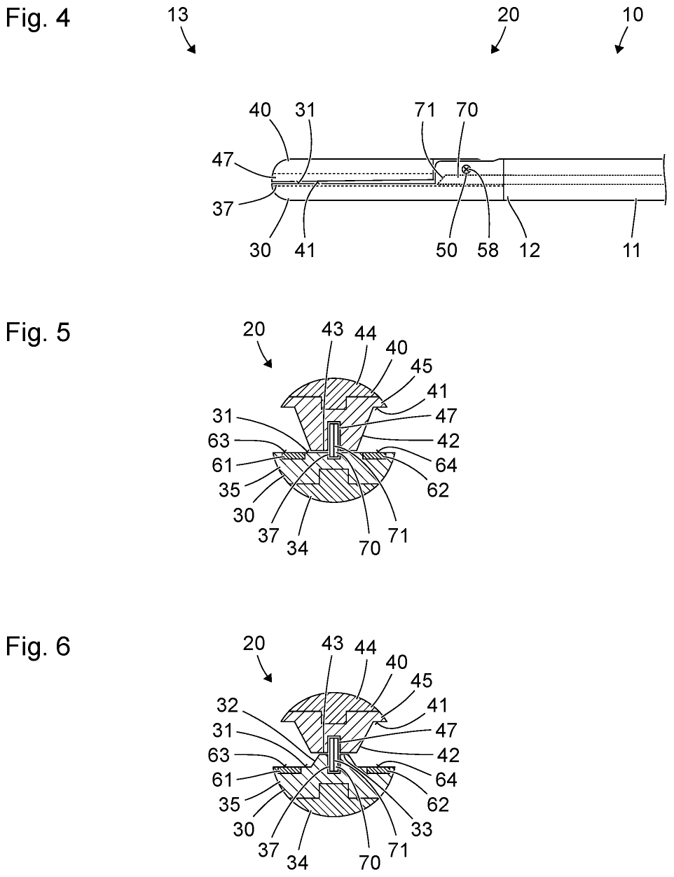

FIG. 4 shows a schematic illustration of a distal end 13 of a further bipolar electrosurgical instrument 10, which may be similar to the instrument illustrated by way of FIGS. 1 to 3 in some features, properties and functions. The type of illustration in FIG. 4 is the same as that in FIGS. 1 and 2. Described below are in particular features, properties and functions of the instrument 10 shown in FIG. 4, where this differs from the instrument illustrated by way of FIGS. 1 to 3.

The instrument 10 shown in FIG. 4 includes a bipolar electrosurgical tool 20 at its distal end 13 that differs from the bipolar electrosurgical tool illustrated by way of FIGS. 1 to 3 on the one hand by a somewhat different layout of the area around the joint 50, and on the other primarily by the arrangement of the gripping faces 31, 41. In the situation shown in FIG. 4, the gripping faces 31, 41 of the jaw parts 30, 40 are only in contact with one another at the distal end of the jaw parts 30, 40. The gripping faces 31, 41 form a small angle, comprising a few degrees. This has the effect that when tissue is arranged and compressed between the gripping faces 31, 41, unlike in the exemplary embodiment of FIGS. 1 to 3, the thickness of this tissue increases to less of an extent from the proximal towards the distal direction, or is even constant.

FIG. 5 shows a schematic illustration of a section through jaw parts 30, 40 of a further bipolar electrosurgical tool, which is similar to the tools illustrated by way of FIGS. 1 to 4 in some features, properties and functions. The type of illustration, in particular the plane of section, corresponds to that in FIG. 3. Described below are in particular features, properties and functions of the bipolar electrosurgical tool 20 shown in FIG. 5, where this differs from the tools illustrated by way of FIGS. 1 to 4.

The cross section of the first jaw part 30 of the bipolar electrosurgical tool 20 shown in FIG. 5 is largely similar to the cross section illustrated by way of FIG. 3. In the illustrated example, the cross section of the first jaw part 30 is simply somewhat flatter, and the groove 37 in the first jaw part 30 is shallower.

Similarly to the example illustrated by way of FIG. 3, the second jaw part 40 includes a first component 44 made from a metal that makes the second jaw part 40 more rigid. Similarly to the example illustrated by way of FIG. 3, the whole of the second gripping face 41 on the second jaw part 40—including the convex region 42—is formed by a component 45 made from an electrically insulating material.

The second jaw part 40 of the bipolar electrosurgical tool 20 shown in FIG. 5 differs from that illustrated by way of FIGS. 3 in that the second gripping face on the second jaw part 40 has a convex region 42 that projects in the direction of the first jaw part 30. In the illustrated example, this convex region 42 forms the majority of the second gripping face 41 on the second jaw part.

The electrically insulating second component 45 of the second jaw part 40 has a cross section with a trapezoidal portion that forms the convex region 42 of the second gripping face 41. The oblique flanks of the convex region 42 of the second gripping face 41—that is to say the two sides of the trapezoidal portion of the cross section of the electrically insulating second component 45 that are not parallel—form an angle of approximately 20°. The sub-region 43 of the convex region 42 of the second gripping face 41 on the second jaw part 40, which is in the form of a flat area and faces the first jaw part 30 and, in the position or situation shown in FIG. 5, abuts against the first gripping face 31 or is opposite it at a small spacing, is narrow enough not to overlap or come into contact with the electrode faces 63, 64 even in the position or situation shown in FIG. 5, but to be spaced apart from them. Tissue that is arranged and compressed or squeezed between the gripping faces 31, 41 of the jaw parts 30, 40 is thus not squeezed in the region of the electrode faces 63, 64, or only to a small extent, and has large cross sections resulting in low current densities.

The flat-area sub-region 43 of the convex region 42 is planar or substantially planar and lies opposite the first gripping face 31, parallel or substantially parallel to it. When the tool 20 is used as intended, tissue is compressed between the gripping faces 31, 41 under an intended closing force of the jaw parts 30, 40 with mechanical properties within a predetermined range. During this intended use, tissue is markedly compressed substantially only between the flat-area sub-region 43 of the convex region 42 and the first gripping face 31, and a sufficiently high current density flows through the tissue and heats it sufficiently for it to be fused or cauterised or sealed by electrosurgery. Below the flat-area region 43, the tissue is not compressed, or is compressed to a markedly lesser extent. Accordingly, the current density and heating there are markedly lower, and the current has no or substantially no electrosurgical effect. Thus, the width of the flat-area sub-region determines the width of the sealing region or sealing strip within which the tissue is sealed. The width of the flat-area sub-region is in particular in the range from 1.6 mm to 2.4 mm.

The above-mentioned groove 47 in which the scalpel 70 is guided with low play and low friction is provided in the electrically insulating second component 45 of the second jaw part 40. In the illustrated example, the groove 47 in the second component 45 of the second jaw part 40 is markedly deeper than the groove 37 in the second component 35 of the first jaw part 30, with the result that the scalpel 70 is guided predominantly in the second jaw part 40.

The whole of the electrically insulating second component 45 of the second jaw part 40 or the convex region 42 has a low thermal conductivity, in order to reduce the dissipation of heat from the sealing region or sealing strip and to increase the electrosurgical effect in the sealing region. The same applies to the whole of the electrically insulating second component 35 of the first jaw part 30 or its region between the electrode components 61, 62.

The whole of the electrically insulating second component 45 of the second jaw part 40 or the convex region 42 comprises in particular silicone rubber, silicone elastomer or silicone resin, and has a Shore A hardness in the range from 60 to 80.

FIG. 6 shows a schematic illustration of a section through jaw parts 30, 40 of a further bipolar electrosurgical tool 20, which is similar to the tools illustrated by way of FIGS. 1 to 5 in some features, properties and functions. The type of illustration, in particular the position and orientation of the plane of section shown in FIG. 6, corresponds to that in FIGS. 3 and 5. Described below are in particular features, properties and functions of the bipolar electrosurgical tool 20 shown in FIG. 6, where this differs from those illustrated by way of FIGS. 1 to 5.

The bipolar electrosurgical tool 20 shown in FIG. 6 differs from the tool illustrated by way of FIG. 5 in particular in that not only has the second gripping face 41 on the second jaw part 40 a convex region 42, but the first gripping face 31 on the first jaw part 30 also has a convex region 32. In the example illustrated in FIG. 6, the convex region 32 of the first gripping face 31 on the first jaw part 30 is formed by a portion, of trapezoidal cross section, of the electrically insulating second component 35 of the first jaw part 30. In the illustrated example, the convex regions 32, 42 of the gripping faces 31, 41 on the jaw parts 30, 40 have different heights and different widths. In particular, a planar flat-area sub-region 43 of the convex region 42 of the second gripping face 41 on the second jaw part 40, facing the first jaw part 30, is wider than a planar flat-area sub-region 33 of the convex region 32 of the first gripping face 31 on the first jaw part 30, facing the second jaw part 40.

The convex region 32 of the first gripping face 31 on the first jaw part 30 is spaced apart from both electrodes 61, 62. Tissue that is arranged between the jaw parts 30, 40 and compressed between the convex regions 32, 42 is not compressed or is only slightly compressed in the area around the electrode faces 63, 64. For this reason, there are markedly larger cross sections and markedly smaller current densities in the area around the electrode faces 63, 64 than between the convex regions 32, 42. As a result, the electrosurgical effect on the tissue is concentrated substantially on the region between the convex regions 32, 42.

The flat-area sub-region 33 of the convex region 32 of the first gripping face 31 and the flat-area sub-region 43 of the convex region 42 of the second gripping face 41 are each planar or substantially planar, and lie parallel or substantially parallel to one another. In the illustrated example, the flat-area sub-region 33 of the convex region 32 of the first gripping face 31 is narrower than the flat-area sub-region 43 of the convex region 42 of the second gripping face 41. The width of the sealing region or sealing strip within which the tissue is sealed corresponds substantially to the width of the flat-area sub-region 33 of the convex region 32 of the first gripping face 31.

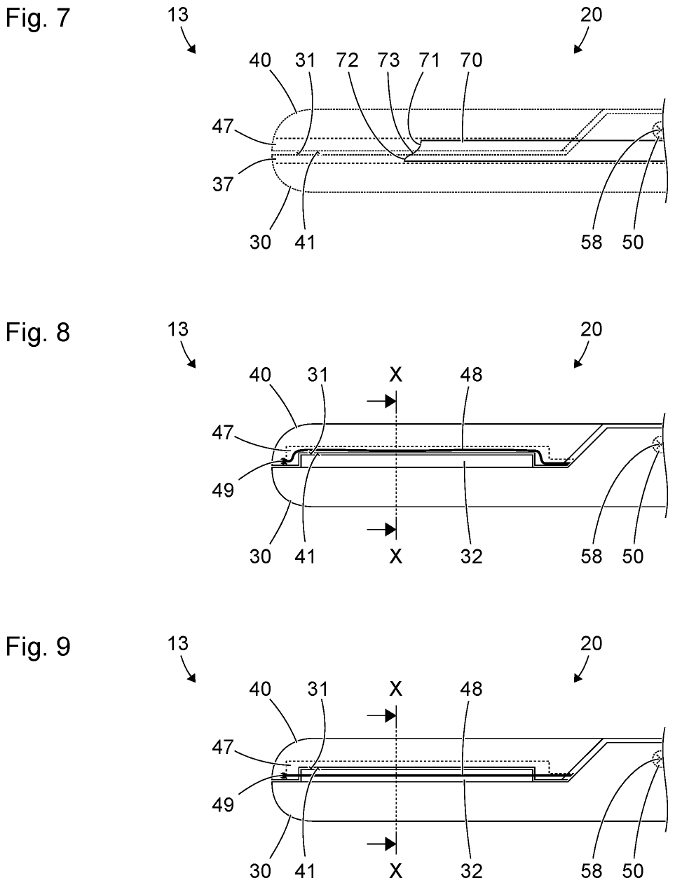

FIG. 7 shows a schematic illustration of a tool 20 at a distal end 13 of a further bipolar electrosurgical instrument, which may be similar to the instruments illustrated by way of FIGS. 1 to 6 in some features, properties and functions. The type of illustration in FIG. 7 corresponds largely to that in FIGS. 1, 2 and 4, but is on a larger scale than these. Further, the jaw parts 30, 40 are illustrated in broken lines and the scalpel 70 in solid lines in order to highlight the properties of the scalpel 70. Described below are in particular features, properties and functions of the tool 20 shown in FIG. 7, where this differs from the tools illustrated by way of FIGS. 1 to 6.

The tool 20 shown in FIG. 7 differs from the tools illustrated by way of FIGS. 1 to 6 in particular in that the blade 71 of the scalpel 70 is not straight but curved or bent. The blade 71 is S-shaped and lies in particular in a plane parallel to the plane of the drawing in FIG. 7. At its ends, which are arranged and guided in the grooves 37, 47 in the jaw parts 30, 40, the blade 71 has steep portions 72, in which the blade is at a right angle or almost at a right angle to the direction of movement intended for the scalpel 70. In a central region, the blade 71 has a flat portion 73 in which the blade 71 extends at an acute angle (in the illustrated example, approximately 45 degrees) to the direction of movement intended for the scalpel 70. When the jaw parts 30, 40 are almost closed, or at a small spacing, as indicated in FIG. 7, the flat portion 73 of the blade 71 can cut tissue that is held and squeezed by the jaw parts.

In contrast to the illustration in FIG. 7, the blade 71 may have a steep portion 72 at only one end and/or may have one or more corners between straight or curved portions.

If the blade has a short overall length and hence an action close to the distal end 13 of the instrument and the tool 20, the non-straight shape of the blade 71 shown in FIG. 7 may simultaneously enable good guidance of the scalpel in the grooves 37, 47 and facilitate cutting using the flat portion 73.

FIG. 8 shows a schematic illustration of a tool 20 at a distal end 13 of a further bipolar electrosurgical instrument, which may be similar to the instruments illustrated by way of FIGS. 1 to 7 in some features, properties and functions. The type of illustration in FIG. 8 corresponds largely to that in FIG. 7. Described below are in particular features, properties and functions of the tool 20 shown in FIG. 8, where this differs from the tools illustrated by way of FIGS. 1 to 7.

The tool 20 shown in FIG. 8 differs from the tools illustrated by way of FIGS. 1 to 7 in particular in that a scalpel for mechanically transecting tissue that is squeezed between the jaw parts 30, 40 and fused, cauterised or sealed is not provided. Instead, the tool 20 includes a cutting wire 48 on the second jaw part 40. The distal end of the cutting wire 48 is secured with high tensile strength to a distal securing point 49 close to the distal end of the second jaw part. In the position shown in FIG. 8, the cutting wire 48 lies in a groove 47 in the second jaw part 40.

In the illustrated example, the first gripping face 31 on the first jaw part 30 has a convex region 32, similar to that in the case of the tool illustrated by way of FIG. 6. The convex region 32 does not extend over the entire length of the first jaw part 30. Instead, the proximal end of the convex region 32 is spaced apart somewhat from the proximal end of the first gripping face 31, and the distal end of the convex region 32 is spaced apart somewhat from the distal end of the first gripping face and the distal end of the first jaw part 30.

In the longitudinal direction, the second jaw part 40 is in the shape of a flat U that embraces the convex region 32 of the first gripping face 31. In the illustrated example, the longitudinal section of the convex region 32 is rectangular, and the flat U shape of the second jaw part 40 is likewise rectangular. As a result, the groove 47 in the second jaw part 40 is not straight in the longitudinal direction either, but is in the shape of a flat rectangular U. The distal securing point 49 of the cutting wire 48 is arranged in the groove 47, close to the distal end thereof. In the closed position shown in FIG. 8, the distal securing point 49 of the cutting wire 48 lies distally in relation to the distal end of the convex region 32 of the first gripping face 31.

FIG. 9 shows a further schematic illustration of the tool 20 from FIG. 8. The type of illustration in FIG. 9 corresponds to that in FIG. 8.

In FIG. 9, the tool 20 is shown in a further position that (taking the position shown in FIG. 8 as a starting point) is achieved by pulling the proximal end of the cutting wire 48 in the proximal direction. As a result, the cutting wire 48 is pulled taut, and largely comes out of the groove 47 and adopts a straight shape, shown in FIG. 9. In this straight shape, the cutting wire 48 lies in a longitudinal groove in the convex region 32 of the first gripping face 31 of the first jaw part 30.

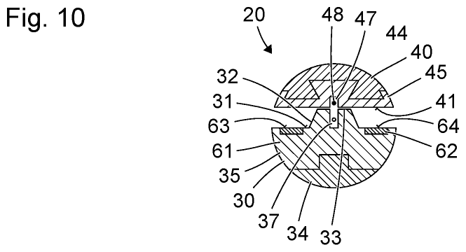

FIG. 10 shows a schematic illustration, on a larger scale, of a cross section along the plane X-X indicated in FIGS. 8 and 9, at a right angle to the planes of the drawing in FIGS. 8 and 9.

In this cross section, the convex region 32 of the first gripping face 31 of the first jaw part 30 and the grooves 37, 47 in the gripping faces 31, 41 can be seen. The cutting wire 48 is shown as a solid black circle in the position illustrated by way of FIG. 8—that is to say within the groove 47 in the second gripping face 41—and as a blank circle in the position illustrated by way of FIG. 9—that is to say within the groove 37 in the first gripping face 31.

As the cutting wire 48 is pulled taut, and as the transition occurs from the position illustrated by way of FIG. 8 to the position illustrated by way of FIG. 9—that is to say during movement of the cutting wire from the groove 47 in the second gripping face 41 into the groove 37 in the first gripping face 31—the cutting wire can, as an electrosurgical tool, transect by electrosurgery tissue that is gripped and squeezed between the gripping faces 31, 41 of the jaw parts 30, 40. During this, the cutting wire can be used as a monopolar or a bipolar electrosurgical tool.

While specific embodiments of the invention have been shown and described in detail to illustrate the application of the principles of the invention, it will be understood that the invention may be embodied otherwise without departing from such principles.

### LIST OF REFERENCE NUMERALS

10 Bipolar electrosurgical instrument
11 Shaft of the bipolar electrosurgical instrument 10
12 Distal end of the shaft 11
13 Distal end of the bipolar electrosurgical instrument 10
20 Bipolar electrosurgical tool
30 First jaw part or first limb of the tool 20
31 First gripping face on the first jaw part 30, facing the second jaw part 40
32 Convex region of the first gripping face 31
33 Flat-area sub-region of the convex region 32
34 Metal first component of the first jaw part 30
35 Electrically insulating second component of the first jaw part 30
37 Groove in the first gripping face 31
40 Second jaw part or second limb of the tool 20
41 Second gripping face on the second jaw part 40, facing the first jaw part 30
42 Convex region of the second gripping face 41
43 Flat-area sub-region of the convex region 42
44 Metal first component of the second jaw part 40
45 Electrically insulating second component of the second jaw part 40
47 Groove in the second gripping face 41
48 Cutting wire on the second jaw part 40
49 Distal securing point of the cutting wire 48
50 Joint between the second jaw part 40 and the first jaw part 30
58 Pivot axis of the second jaw part 40, defined by the joint 50
61 First electrode component in the first gripping face 31
62 Second electrode component in the first gripping face 31
63 Electrode face on the first electrode component 61
64 Electrode face on the second electrode component 62
70 Scalpel of the tool 20, movable in the grooves 37, 47
71 Blade of the scalpel 70
72 Steep portion of the blade 71
73 Flat portion of the blade 71

1. A bipolar electrosurgical tool for a bipolar electrosurgical instrument for closing a hollow organ or sealing other tissue, the bipolar electrosurgical tool comprising:
a first jaw part with a first gripping face,
a second jaw part with a second gripping face facing the first gripping face;
a joint that enables the second jaw part to move pivotally in relation to the first jaw part;
a first electrode on the first jaw part; and
a second electrode on the first jaw part, wherein the second electrode is electrically insulated from the first electrode and there is no electrode arranged on the second jaw part.

2. (canceled)

3. The bipolar electrosurgical tool according to claim 1, wherein the entire second gripping face is configured to be electrically insulating.

4. The bipolar electrosurgical tool according to claim 1, further comprising:
a cutting device configured to be moved between the first jaw part and the second jaw part, for mechanically transecting tissue that is held between the first jaw part and the second jaw part.

5. The bipolar electrosurgical tool according to claim 1, wherein, the first electrode and the second electrode are arranged next to one another and parallel to one another on the first gripping face.

6. The bipolar electrosurgical tool according to claim 1, further comprising:
a convex surface region between the first electrode and the second electrode, which projects in a direction of the second jaw part.

7. The bipolar electrosurgical tool according to claim 6, wherein the convex surface region is formed by a resilient component.

8. The bipolar electrosurgical tool according to claim 1, further comprising:
a convex surface region on the second gripping face of the second jaw part, wherein the convex surface region projects towards the first gripping face of the first jaw part.

9. The bipolar electrosurgical tool according to claim 8, in which the convex surface region on the second gripping face of the second jaw part is arranged and configured such that the convex surface region on the second gripping face of the second jaw part cannot come into contact with either the first electrode or the second electrode, even in a closed condition of the tool.

10. The bipolar electrosurgical tool according to claim 8, wherein the convex surface region on the second gripping face of the second jaw part is formed by a component made of a resilient material.

11. A bipolar electrosurgical instrument comprising:
a shaft,
a bipolar electrosurgical tool, for closing a hollow organ or sealing other tissue, the bipolar electrosurgical tool comprising:
a first jaw part with a first gripping face;
a second jaw part with a second gripping face facing the first gripping face;
a joint that enables the second jaw part to move pivotally in relation to the first jaw part;
a first electrode on the first jaw part; and
a second electrode on the first jaw part, wherein the second electrode is electrically insulated from the first electrode and there is no electrode arranged on the second jaw part, and wherein the bipolar electrosurgical tool is connected or connectable to a distal end of the shaft.

12. The bipolar electrosurgical tool according to claim 1, wherein, when used as intended, a surface area of the first electrode and a surface area of the second electrode are each greater than a cross section of tissue that is gripped or squeezed between the jaw parts during an intended electrosurgical treatment.

13. The bipolar electrosurgical tool according to claim 1, wherein, when used as intended, a width of the first electrode in strip form and a width of the second electrode in strip form are each greater than a thickness of tissue that is gripped or squeezed between the jaw parts during an electrosurgical treatment intended.

14. The bipolar electrosurgical tool according to claim 1, wherein:
the first electrode and the second electrode are arranged on or close to edges of the first gripping face that face away from one another; and
the first gripping face between the first electrode and the second electrode is configured to be completely electrically insulating.

15. The bipolar electrosurgical tool according to the preceding claim 6, wherein the convex surface region has a web shape or a bead shape with a substantially rectangular, trapezoidal or rounded cross section.

16. The bipolar electrosurgical tool according to claim 15, wherein the convex surface region is formed by a resilient component.

17. The bipolar electrosurgical tool according to claim 9, wherein the convex surface region on the second gripping face of the second jaw part is formed by a component made of a resilient material.

18. The bipolar electrosurgical tool according to one of claim 8, wherein the convex surface region on the second gripping face has a web shape or a bead shape with a substantially rectangular, trapezoidal or rounded cross section.

19. The bipolar electrosurgical tool according to claim 18, wherein the convex surface region on the second gripping face of the second jaw part is formed by a component made of a resilient material.

20. The bipolar electrosurgical tool according to one of claim 9, wherein the convex surface region on the second gripping face has a web shape or a bead shape with a substantially rectangular, trapezoidal or rounded cross section.

21. The bipolar electrosurgical tool according to claim 20, wherein the convex surface region on the second gripping face of the second jaw part is formed by a component made of a resilient material.
